(19) United States
(12) Patent Application Publication
(10) Pub. No.: US 2023/0280477 A1
(43) Pub. Date: Sep. 7, 2023

(54) System for Calculating Highly Accurate GPS Location for Mobile Devices

(71) Applicant: Rohit Seth, Ontario (CA)

(72) Inventor: Rohit Seth, Ontario (CA)

(21) Appl. No.: 17/963,132

(22) Filed: Oct. 10, 2022

Related U.S. Application Data
(63) Continuation of application No. PCT/US21/26913, filed on Jan. 1, 0001.
(60) Provisional application No. 63/008,632, filed on Apr. 10, 2020.
(60) Provisional application No. 63/013,479, filed on Apr. 21, 2020.

(51) Int. Cl. G01S 19/48; G01S 19/40; G01S 19/14

(52) U.S. Cl. CPC G01S 19/48; G01S 19/40; G01S 19/14

(57) ABSTRACT

A system includes Referential Global Positioning System (RGPS) base stations, servers and mobile devices. Each RGPS station includes: (i) global navigation satellite system (GNSS) receivers and antennas for receiving GNSS geolocation data, Cellular Location Technology (CLT) geolocation data, Wi-Fi Positioning System (WPS) geolocation data; (ii) a data processor for positioning error correction, and signal processing; and (iii) a transmitter for transmitting the error corrections and processed GPS data to the servers. The servers (i) receive, aggregate, and store, the GNSS, CLT, and Wi-Fi error corrections and processed GPS data, and (ii) transmit the location-specific GPS data to any mobile device in proximity. Each mobile device calculates a calculate a highly accurate location of the mobile device by obtaining and combining the location-specific GPS data from a RGPS server, and device-specific geolocation data from a GNSS sensor, a CLT sensor and a WPS sensor, on the mobile device.

### CROSS-REFERENCE TO RELATED APPLICATIONS

This application is a continuation of PCT Application No. PCT/IB2021/000256, entitled “System for Calculating Highly Accurate GPS Location for Mobile Devices,” which claims the benefit of U.S. Provisional Patent Application No. 63/008,632 filed on Apr. 10, 2020, entitled “High-Accuracy GPS and Related Functionality for Mobile Devices,” and U.S. Provisional Patent Application No. 63/013,479 filed on Apr. 21, 2020, entitled “Methods of Geospatial Technology for Disease Prevention,” each of which is incorporated by reference herein in their entirety.

This application is related to U.S. Pat. No. 10,852,143, filed on Jun. 26, 2019 entitled “Motion Sensor with Drift Correction,” the contents of which are incorporated by reference herein it its entirety.

### FIELD OF THE INVENTION

Some implementations relates generally to the field of mobile device location identification, and more particularly to implementation of a high-accuracy GPS system on networked mobile devices, and to related applications of such functionality.

### BACKGROUND OF THE INVENTION

The Global Positioning System (GPS) is a satellite-based navigation system made up of a network of satellites placed in orbit. GPS satellites circle the Earth and continually transmit messages to Earth that include the time the message was transmitted and the satellite position at the time of the message transmission. A GPS receiver uses the information from the messages it receives from multiple satellites to determine (calculate) the location of the GPS receiver. A GPS receiver must acquire signals from at least four satellites to reliably calculate a three-dimensional position. Ideally, these satellites should be distributed across the sky. The receiver performs mathematical calculations to establish the distance from a satellite, which in turn is used to determine its position.

Theoretically, GPS can provide worldwide, three-dimensional positions, 24 hours a day, in any type of weather. However, atmospheric conditions can cause errors to the signal from the satellite, which can affect the calculated positional data. The system does have some additional limitations. There must be a relatively clear “line of sight” between the receiver's GPS antenna and four or more satellites. Objects, such as buildings, overpasses, and other obstructions, that shield the antenna from a satellite can potentially weaken a satellite's signal such that it becomes too difficult to ensure reliable positioning. These difficulties are particularly prevalent in urban areas where the GPS signal may bounce off nearby objects causing interference and errors.

### SUMMARY

Some implementations described herein respond to unmet needs in the market by introducing a hardware and software system that allows most of the current smartphones and mobile devices worldwide (fitted with consumer-grade GPS chips) to wirelessly receive GPS-error-correction data generated from the nearest base station(s) from a Referential Global Positioning System (RGPS) network, enabling such mobile devices to achieve sub-meter GPS location accuracy, and unlocking other novel functionalities that take advantage of such improved location accuracy. The system according to some implementations is relatively inexpensive, is backward compatible to most current smartphones and mobile devices worldwide, does not require the user to buy additional hardware, and is implemented, on the user-side, through a software application (app) that can be installed from an app store.
(A1) A system comprising a plurality of Referential Global Positioning System (RGPS) base stations (e.g., some static, dynamic mobile station) that are connected via a network, wherein each RGPS station comprises (i) one or more global navigation satellite system (GNSS) receivers and antennas configured to receive geolocation data from satellites, Cellular Location Technology (CLT) geolocation data from cellular networks, Wi-Fi Positioning System (WPS/WiPS/WFPS) geolocation data from mobile hotspots or Wireless Access Points; (ii) a data processor configured to perform positioning error correction, and signal processing, on the cumulative geolocation data to form a high-accuracy Global Positioning System (GPS); (iii) a transmitter configured to transmit error corrections and processed GPS data to one or more servers coupled to the network; the one or more servers configured to (i) receive, aggregate, and store, the GNSS, CLT, and Wi-Fi error corrections and processed GPS data, and (ii) transmit the location-specific GPS data to any mobile device in proximity; and one or more mobile devices, each mobile device configured to (i) receive the location-specific GPS data from a RGPS server in proximity to the mobile device, (ii) obtain device-specific geolocation data from a GNSS sensor on the mobile device, (iii) obtain device-specific geolocation data from a CLT sensor on the mobile device, (iv) obtain device-specific geolocation data from a WPS sensor on the mobile device and (v) combine the location-specific GPS data with the device-specific GPS data to calculate a highly accurate location of the mobile device.
(A2) The system of (A1-A2), wherein at least some of the RGPS base stations are positioned at locations where there is no reception or only partial reception of GPS signals from the respective networks, and wherein the locations are in proximity to predetermined locations of the one or more mobile devices.
(A3) The system of claim (A1-A2), wherein the locations are at or near street-level close to streets where the one or more mobile devices are known to be present.
(A4) The system of any of claims (A1-A3), wherein the one or more servers or the plurality of RGPS base stations include a wireless communication subsystem to wirelessly transmit the location-specific GPS data to any mobile device in proximity.
(A5) The system of any of claim (A1-A4), wherein each RGPS base station is configured to receive the GNSS signal data from a plurality of satellites, calculate coordinates for a location, and compare the calculated coordinates to predetermined coordinates, to generate a compensation value or an error correction data for the location.
(A6) The system of any of claim (A1-A5), wherein each RGPS base station is configured to receive the CLT signal data from a plurality of cellular network signals, calculate coordinates for a location, and compare the calculated coordinates to predetermined coordinates to generate a compensation value or an error-correction data for the location.
(A7) The system of any of claim (A1-A6), wherein each RGPS base station is configured to receive the WPS signal data from a plurality of network devices, calculate coordinates for a location, and compare the calculated coordinates to predetermined coordinates, to generate a compensation value or an error correction data for the location.
(A8) The system of claim (A1-A7), wherein each RGPS base station is further configured to receive the GNSS, CLT, WPS signal data, calculate the coordinates for the location, and compare the calculated coordinates to predetermined coordinates, on a continuous and periodic basis.
(A9) The system of any of claims (A1-A8), wherein each RGPS base station is further configured to receive the GNSS, CLT, WPS signal data, calculate the coordinates for the location, and compare the calculated coordinates to predetermined coordinates, for different pre-determined times during the day.
(A10) The system of any of claims (A1-A9), wherein each RGPS base station is configured to transmit respective base station identification data that includes a respective identifier, a location, date and time of the error correction, or identifiers of location of the GNSS satellites, CLT network towers, WPS network devices, and wherein the one or more servers are configured to receive and use the respective base station identification data to obtain the location-specific GPS data.
(A11) The system of any of claim (A1-A10), wherein each mobile device includes a software application that (i) receives the location-specific GPS data, (ii) obtains the device-specific GPS location data, and (iii) combines the location-specific GPS data with the device-specific GPS data to calculate the location of the mobile device.
(A12) The system of claim (A1-A11), wherein the software application is further configured to establish a data connection with the one or more servers, transmit an approximate or non-corrected GPS location of the mobile device to the one or more servers, and receive a relevant error correction and processed GPS data generated by a base station that is most proximate to the mobile device's current location.
(A13) The system of any of claims (A1-A12), wherein each mobile device includes a software application that (i) stores the location of the mobile device on the mobile device and (ii) provides the location to one or more other applications on the mobile device.
(A14) The system of any of claims (A1-A13), wherein each mobile device is further configured to transmit the location of the mobile device to the one or more servers, and wherein the one or more servers is further configured to receive the location of the mobile device to update the location-specific GPS data.
(A15) The system of any of claims (A1-A14), wherein the one or more servers are further configured to (i) receive location of the mobile device from the mobile device, and (i) track locations of the one or more mobile devices based on the location of the mobile device. In some implementations, location data may be anonymous or provided anonymously.
(A16) The system of any of claims (A1-A15), wherein the one or more servers are further configured to generate an alarm if it is determined that any of the one or more mobile devices are closer than a predetermined distance.
(A17) The system of any of claims (A1-A16), wherein the one or more servers are further configured to generate and transmit an alarm, to the one or more mobile devices or to a third-party device distinct from the one or more mobile devices, if it is determined that any of the one or more mobile devices are close to a predetermined location.
(A18) The system of any of claims (A1-A17), wherein the one or more servers are further configured to anonymize any identifiable information received from the one or more mobile devices before transmitting any notifications to the one or more mobile devices, or a third-party device distinct from the one or more mobile devices.
(A19) The system of any of claims (A1-A18), wherein the one or more servers are further configured to track locations of the one or more mobile devices to determine a set of devices of the one or more mobile device that may be within a given proximity to another device.
(A20) The system of any of claims (A1-A19), wherein the one or more mobile devices include a motion sensor with drift correction configured to track precision location and orientation of a mobile device regardless of external reference markers, transponders, or satellites, and wherein the one or more mobile devices are further configured to fuse the precision location and orientation with the location-specific GPS data and the device-specific GPS data to calculate the location of the mobile device.
(A21) The system of any of claims (A1-A20), wherein the one or more mobile devices are configured to use Bluetooth as a metric to determine proximity to another device in addition to GPS.
(A22) The system of any of claims (A1-A21), wherein the one or more mobile devices are configured to use a geo-coding format for distancing queries and zone classification in three dimensions.
(A23) The system of any of claims (A1-A22), wherein the one or more mobile devices are configured to use anonymous tokens or encoding that provides security, and wherein the anonymous tokens include (i) a specification of resolution depth of geospatial data, (ii) an obfuscation or encryption for privacy or security that encrypts any address-specific component, and/or (iii) unencrypted data for number of interactions within a region, times of data, or demographic data.
(A24) The system of any of claims (A1-A23), wherein the one or more servers or the one or more mobile devices are configurable to program or control variables or policies, for minimum distancing, exposure time, or demographic data, during a real-world event.
(A25) The system of any of claims (A1-A24), wherein the one or more servers or the one or more mobile devices are configured to generate visualizations for contact tracing, in a mobile application, showing historical interactions with risky individuals, risk levels, or course of action.

### BRIEF DESCRIPTION OF THE DRAWINGS

For a better understanding of the various described implementations, reference should be made to the Description of Implementations below, in conjunction with the following drawings in which like reference numerals refer to corresponding parts throughout the figures.

FIG. 1 is a schematic diagram of a network environment, in accordance with some implementations.

FIG. 2A is a block diagram illustrating a representative base station system, according to some implementations.

FIG. 2B is a block diagram illustrating a representative server system, according to some implementations.

FIG. 2C is a block diagram illustrating a representative mobile device system, according to some implementations.

FIGS. 3A-3B are a flowchart representation of a system designed to calculate a highly accurate location of a mobile device, according to some implementations.

FIG. 4 illustrates a BIOS system according to some implementations.

FIG. 5 illustrates various measurement sources, individually marked in different colors, on a Google Maps screen capture of the BIOS app, according to some implementations.

FIGS. 6A-6B illustrate representations of how GEOSHASH encodes, according to some implementations.

FIG. 7 illustrates a representation of a sample GEOSHASH string with its resolution components according to some implementations.

FIG. 8 illustrates a representation of a BIOS pandemic prevention mechanism according to some implementations.

FIG. 9 illustrates a representation of an interaction between users according to some implementations.

FIG. 10 is an image of a screen capture of the BIOS mobile app's Interactions Screen according to some implementations.

FIG. 11 illustrates a representation of how interaction data is utilized by a mobile device according to some implementations.

FIG. 12 illustrates a representation of how event data is transmitted between components of the BIOS system according to some implementations.

FIG. 13 illustrates location positioning of the mobile device using the BIOS application, according to some implementations.

### DESCRIPTION OF IMPLEMENTATIONS

Reference will now be made in detail to implementations, examples of which are illustrated in the accompanying drawings. In the following detailed description, numerous specific details are set forth in order to provide a thorough understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

It will also be understood that, although the terms first, second, etc. are, in some instances, used herein to describe various elements, these elements should not be limited by these terms. These terms are only used to distinguish one element from another. For example, a first electronic device could be termed a second electronic device, and, similarly, a second electronic device could be termed a first electronic device, without departing from the scope of the various described implementations. The first electronic device and the second electronic device are both electronic devices, but they are not necessarily the same electronic device.

The terminology used in the description of the various described implementations herein is for the purpose of describing particular implementations only and is not intended to be limiting. As used in the description of the various described implementations and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

High-Accuracy GPS and Related Functionality for Mobile Devices

At this time, state-of-the-art consumer-grade single GPS chips (of the type commonly included with most modern smartphones and other mobile devices) from any leading manufacturer can only achieve location accuracies of approximately 5 to 10 to 15 meters apart. This is barely sufficient and usable for some un-demanding consumer applications, such as navigation by car on roads. Various technological means already exist to achieve better GPS location accuracies (from one to two meters, down to a sub-meter range) by employing various “differential correction of GPS data” protocols. A major drawback of these current high-accuracy GPS systems is the fact that they are bulky, very expensive, power hungry, require proprietary receivers and communication protocols and additional land-based infrastructure; for these reasons, such high-accuracy GPS systems are almost never included with consumer-grade smartphones and other mobile devices.

One such high-accuracy GPS system known in the art is the differential GPS (DGPS). The underlying premise of differential GPS (DGPS) is that any two receivers that are relatively close together will experience similar reception errors for incoming GPS satellite signals. DGPS requires that a GPS receiver be set up on a precisely known location. This GPS receiver is known as the “base station,” the “reference station,” or the “beacon station.” In some implementations, the base station is static or dynamic mobile. The base station receiver calculates its position based on GPS satellite signals and compares this location to the known location. The difference (or instant raw error) is averaged over time, and it is subjected to some processing and then this “error-corrected information” is sent (via various wireless communications protocols) to the second GPS receiver, which is known as the “roving receiver.” This error-correction is then applied to the GPS data independently received from the satellites by the roving receiver, resulting in much improved location accuracy for the roving receiver.

Current “state of the art” DGPS base stations can be made relatively compact (approximately the size of a cereal box), relatively inexpensive, and with low power consumption that makes them suitable even for locations not connected to the electrical grid (with power provided by a small solar panel and an optional battery). A network of DGPS base stations placed even 5 to 10 kilometers (km) apart from each other can still significantly improve the location accuracy of any connected roving DGPS, approaching one meter accuracy; moreover, spacing the DGPS base stations even closer to each other can further improve the location accuracy of roving DGPS receivers; sub-meter location accuracy should be achievable within a city with maybe just one DGPS base station for every couple of blocks, while more GPS-error-prone areas of a city (or areas where accuracy is down to 10-20 centimeters (cm) is desired) could be fitted with a DGPS base station at every street corner or on every lamp post.

Despite the obvious advantage (significantly increased GPS location accuracy) that would result from implementing a dense network of inexpensive DGPS base stations in most cities of the world, this technology has not been widely implemented anywhere in the world at this time, the main reason being that such base stations only work with very expensive and custom-made DGPS roving receivers, which are bulky, power hungry, and require proprietary hardware and communication protocols. As such, DGPS is currently used only by relatively few mission-critical mobile users that absolutely require high-accuracy location and that can afford the high cost of the hardware (purpose-built roving DGPS receivers, mostly vehicle-mounted) and the high monthly subscription costs of this service. Conversely, the limited number of potential mobile DGPS users acts as a disincentive for DGPS providers to expand their network of DGPS base stations to the point where they would become ubiquitous and pervasive in most urban centers; at the same time, billions of smartphones and mobile devices worldwide (otherwise fitted from the factory with consumer-grade GPS chips) are locked out of the current DGPS eco-system because they lack the hardware capability to communicate with and take advantage of any currently installed DGPS base station network. There is an unmet need in the market for a universal implementation that could open access to DGPS for most of the smartphones and mobile devices worldwide (fitted with consumer-grade GPS chips), enabling such mobile devices to achieve sub-meter GPS location accuracy and unlocking other novel functionalities that could take advantage of such improved location accuracy. There is an even larger unmet need in the market for such implementation to be relatively inexpensive, to be universally backward compatible to most current smartphones and mobile devices worldwide, to not require additional hardware, and to ideally be implemented through a user-friendly software application (app) that most users could install from their favorite app store.

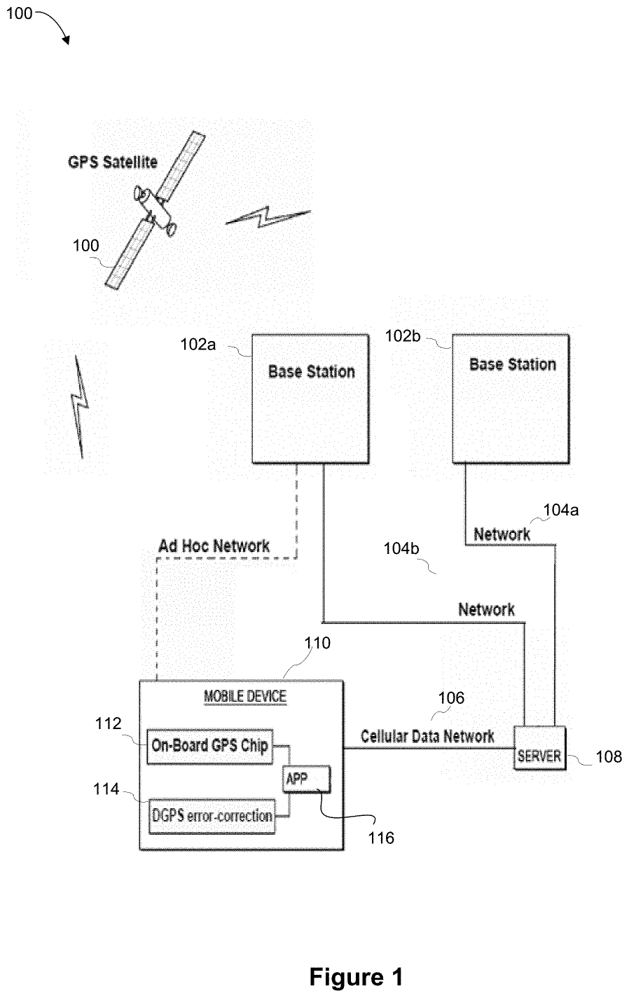

FIG. 1 is a schematic diagram of a network environment or system 100, in accordance with some implementations. In some implementations, the system 100 includes a network of RGPS base stations 102a, 102b; a network server 108; and a software application 116 installed on wireless mobile data-networked devices 110 (e.g., smartphones, etc.) fitted with a consumer-grade GPS chip 112. Each base station 102a, 102b is placed at a fixed terrestrial, aqueous or aerial location with a relatively unobstructed partial view to the sky (possible locations: walls of buildings, lamp posts, roofs, towers, traffic light housings, anchored buoys, light houses, navigational markers, bridges, underpasses, tethered balloons, hovering aircraft, etc.).

In some implementations, each RGPS base station 102a, 102b includes:
a source of electrical power;
one or more GPS receiver chips and antennas to receive data from GPS satellites 100;
data-processing means for performing differential correction and other error-correction and processing steps to the GPS signals received from GPS satellites 100;
networking means (wired or wireless) to transmit such “error-corrected” data 114 and processed GPS data to one or more remote networked servers 108;
wireless networking means to transmit such error-corrected data 114 and processed GPS data by direct wireless (data) connection to any nearby mobile networked devices (smartphones, etc.) fitted with a consumer-grade GPS chip and further fitted with the appropriate software and hardware to receive such data;

In some implementations, the software application 116 receives RGPS error-correction data 114 and processed GPS data for the respective location (via a wireless network 106) from one or more remote network servers (or, optionally, directly from a nearby RGPS base station) and whereby the software application 116 uses such received RGPS error-correction data (also referred to herein as “error corrected data”), together with the GPS location data independently provided by the device's onboard GPS sensor, to determine (calculate) the location of the mobile device 110 with significantly increased accuracy.

Referring to FIG. 1, in some implementations, a network of RGPS base stations 102a, 102b is installed across the target area. Since each individual base station 102a, 102b is relatively compact, inexpensive, and has low power requirements, there is a great multitude of possible places and locations to install them, according to the desired coverage and density. While a relatively unobstructed partial view to the sky is a natural requirement for each RGPS base station 102a, 102b, an implementation would not necessarily place them in locations with ideal or perfect reception of GPS signals from satellites, but instead it would place the RGPS base stations 102a, 102b in such places where they would mimic closely the actual imperfect reception conditions experienced by mobile GPS receiver users in real life, so that the error-corrected GPS data generated by these base stations becomes more relevant and more applicable to end users and their GPS-enabled mobile devices. For example, in an certain “urban canyon” type of scenario of a large city downtown (flanked with high buildings and skyscrapers), it would be more advantageous to not place RGPS base stations on the roofs of high buildings (where near-perfect GPS signal reception would cause such base stations to generate RGPS error correction that is less useful to users at street level), but instead to place such RGPS base stations closer to street level, so as to generate more relevant and useful RGPS error correction for end users (for example, preferred locations would be on mailboxes, traffic signs, hand railings, bike racks, street railings, doorframes of buildings, bus shelters, retail shopping concourses, or even on street garbage bins and recycling bins).

In some implementations, each RGPS base station 102a, 102b (with fixed coordinates) will receive GPS satellite data from a plurality of GPS satellites and will calculate location system coordinates continuously (or at certain intervals), and compare the calculated location system coordinates to the (known) fixed coordinates to generate a compensation value or a RGPS error-corrected data 114, custom generated for each specific location and for each time of the day. Such processing of data can be done either at the level of each RGPS base station 102a, 102b, or at a networked server location.

In some implementations, such RGPS base stations 102a, 102b are data-networked by wired or wireless networking means, enabling them to transmit their location-specific RGPS error-correction data 114 to a network server. The transmitted data may optionally include an identifier of the ground station, a location of the base station, the date and time of calculation of the error-correction value, and identifiers of the location system satellites associated with the GPS satellites 100 used to determine the location system coordinates. The networking means can be any wired or wireless network, or any combination thereof, or any combination of connections and protocols that will support communications between the base stations 102a, 102b and the network server 108 in accordance with some implementations of this invention.

In some implementations, the base stations 102a, 102b might also broadcast error-correction data 114 locally (to the geographic vicinity of the ground station, by any known wireless data transmission protocols or combinations thereof), whereby error-correction data for the specific locale could be received directly by any nearby mobile device equipped with compatible hardware and software, according to the specific networking protocol used.

In some implementations, the network server 108 can be a management server, a web server, or any other electronic device or computing system capable of receiving, sending and/or processing data in accordance with implementations of this invention.

In some implementations, the software application 116 can be any software or program installed on wireless mobile data-networked devices (smartphones, etc.) fitted with a consumer-grade GPS chip. Such application 116 can be offered for download by the user from the app store according to each mobile operating system (such as iOS or Android), or can come pre-installed from the factory, or be a part of the mobile operating system, or be mandate by the appropriate authorities or by court order. One functionality of such application 116 is to establish a “data connection” (preferably via the mobile device's wireless internet connection) with one or more remote network servers, to send to such server an approximate (non-corrected) GPS location of the mobile device at a given time, and, in reply, to receive from the server the relevant “error-corrected” and processed GPS data generated from the base station(s) most proximate to the mobile device's current location.

In some implementations, the application 116 is able to use any other hardware and software (already present on that mobile device, or from attached peripherals), to receive “error-corrected” GPS data directly from a nearby RGPS base station 102a, 102b, by any known means and protocols of direct data transmission to which the host mobile device might be capable.

Another functionality of the application 116, in an implementation, is to use the “error-corrected” RGPS data for that vicinity (received from the server), together with the (less-accurate) GPS location data independently provided by the device's native onboard GPS sensor, to determine (calculate) the error corrected location of the mobile device with significantly increased accuracy. Once this error corrected location value for the mobile device is computed in real time, the app can offer it locally to any other software (already present on that mobile device) that might require GPS coordinates (as an alternative or replacement to the less-accurate values provided by the native GPS chip fitted on that mobile device); the app can also log such data locally, or can upload such location coordinates to the network server (in batches or in real time, for immediate use or for long-term storage), alone or in combination with any other identifiable or anonymized data respecting the unique user and unique mobile device.

In today's world, many individuals are rarely separated from their (data-connected) smartphones and mobile devices, whether at home, at work or elsewhere. Accordingly, the ability to track the precise position of mobile devices translates into an ability to precisely track (at any time and continuously, if needed) the location of the person uniquely attached to/associated with each such device. Once this error corrected RGPS location data (e.g., accurate to 10 cm-50 cm) becomes available to mobile devices in accordance with some implementations, interesting possibilities open up for other value-added functionalities, based on access to real-time or historical (logged) precise location information for a person's smartphone. Below are some example use cases:
Monitoring compliance with, and enforcing judicial “restraining orders” (which are often worded along the following lines: “this person shall not come closer than 50 meters from a target person or place.” The app, in accordance with some implementations, is programmed to provide an alarm, notification or a warning on the user's phone if the app detects that the user's smartphone is physically present at (or near the) restraining distance issued by the court, or even send police immediately (and/or notify the intended target) if it is deemed that such proximity was not accidental.
Other predefined geo-fencing type of schemes where the app can execute certain pre-programmed actions (alarms, messages) when automatically triggered by the mobile device's proximity to certain coordinates.
various movement restrictions imposed by governments on the population in response to pandemics such as COVID-19. For example, “social distancing” orders could be made very easy to enforce or to monitor compliance, by either mandating that such error-corrected location tracking app should be installed on every person's smartphone, or by encouraging voluntary installation of the app in accordance with some implementations of this invention. To alleviate the privacy concerns raised by the use of such app for location tracking of population on a massive scale, various anonymizing schemes and protocols can be implemented at the app level or at the server and storage database level; for example, raw data containing location tracking for populace could be securely stored in “fully identifiable” format, but only made available for processing in an “anonymized” format. If the app detects that two individuals are physically coming closer to each other than the 2 meter limit imposed by the government, the app could first issue a warning based on anonymized location data, but if any further or more prolonged physical closeness occurs between the same two individuals, the app could be allowed to access “fully identifiable” information (real time and/or historical) about the location of such individuals, to be communicated to law enforcement or to health authorities if they need to locate and pursue the flagged individuals. Similarly, a healthcare worker could have her location monitored in real time, and she could, for example, receive a notification about her proximity to a high-risk area or proximity to any infected or high-risk individuals. In other scenarios, if an individual is suspected or confirmed as a carrier of a transmissible infection, with or without that individual's permission, recent historical location data for that individual could be retrieved, graphed or processed, so as to generate a list of all other individuals who ever came close to that individual in the previous two weeks, thus enabling informed decisions as to how to deal with the situation, together with immediate notification capabilities for the at-risk individuals flagged by the app in accordance with some implementations of this invention.

Many other possible functionalities of the app in accordance with implementations of this invention are possible within the implementations described herein, and inventor claims as part of this invention all such previously known or envisaged applications or scenarios that are rendered more feasible or significantly improved by the high-precision location capabilities enabled by the use of this invention.

While the foregoing written description of the invention enables one of ordinary skill to make and use what is considered presently to be the best mode thereof, those of ordinary skill will understand and appreciate the existence of variations, combinations, and equivalents of the specific implementations, method, and examples herein. The invention should therefore not be limited by the above-described implementations, method, and examples, but by all implementations and methods within the scope and spirit of the invention.

In some implementations, several base stations, acting as wireless communication nodes organized in a mesh topology, may also be configured as a wireless mesh network among base stations, to effect improved error correction; in further optional implementations, the app according to this invention may additionally cause the mobile devices (roving receivers) themselves to join such wireless mesh network (on an ad hoc basis or otherwise) to effect further-improved location accuracy; for even further improvements in location accuracy (especially useful in indoor or other challenging environments), other optional implementations of this invention may additionally combine the features described above with various other known positioning system protocols and helper solutions to further reduce GPS error, such as telemetry by Wi-Fi, Internet-of-Things (IoT) sensors, and/or solutions based on Radio Frequency Identifiers (RFID), Radio Frequency (RF), Bluetooth, and Ultra-Wideband (UWB), and/or other technologies based on Wi-Fi, magnetic field, motion sensors, Inertial Measurement Units (IMUs) and vision techniques.

Particularly useful and novel functionalities can be achieved by combining the features of implementations described herein with the teachings and claims of related U.S. Patent Application Publication No. 2020/0011669, titled “Motion Sensor with Drift Correction” and filed on Jun. 26, 2019, by the same inventor Rohit Seth, the entire content of which reference is specifically incorporated herein in its entirety.

Particularly, improved, useful and novel functionalities will result when the base stations (and, optionally, the mobile devices (roving receivers)), according to some implementations, additionally adopt and implement the methods, systems, and devices described in U.S. Patent Application Publication No. 2020/0011669, which teaches motion sensors with drift correction, capable of tracking the precise location and orientation of an object independent of external reference markers, transponders, or satellites. The fusing (at hardware or software level) of location data generated by RGPS (according to the above description) with the accurate tracking data generated by the tracking devices/methods described in U.S. Patent Application Publication No. 2020/0011669 typically results in further improvements in location accuracy (especially useful in indoor or other challenging/complex environments such as hospitals, airports, shopping malls, etc.).

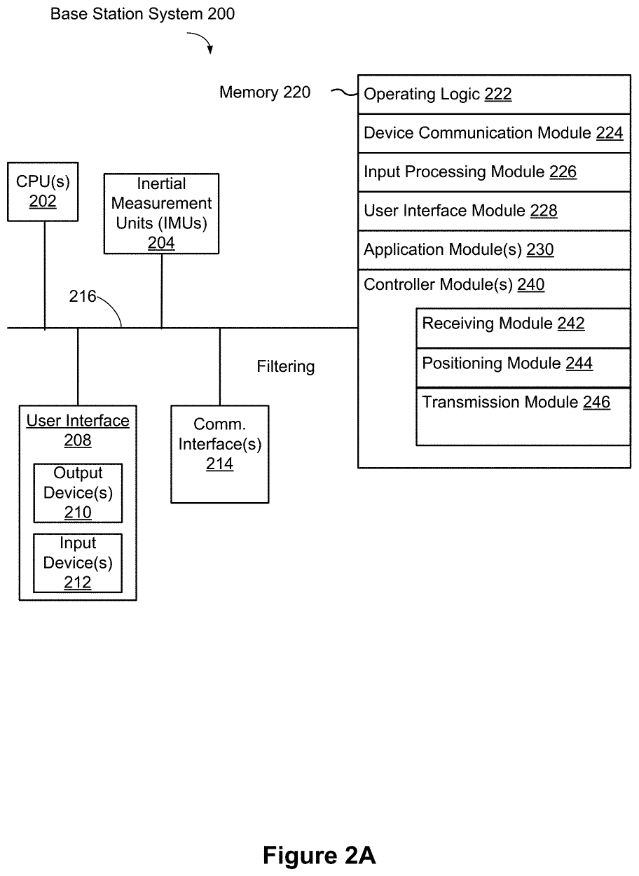

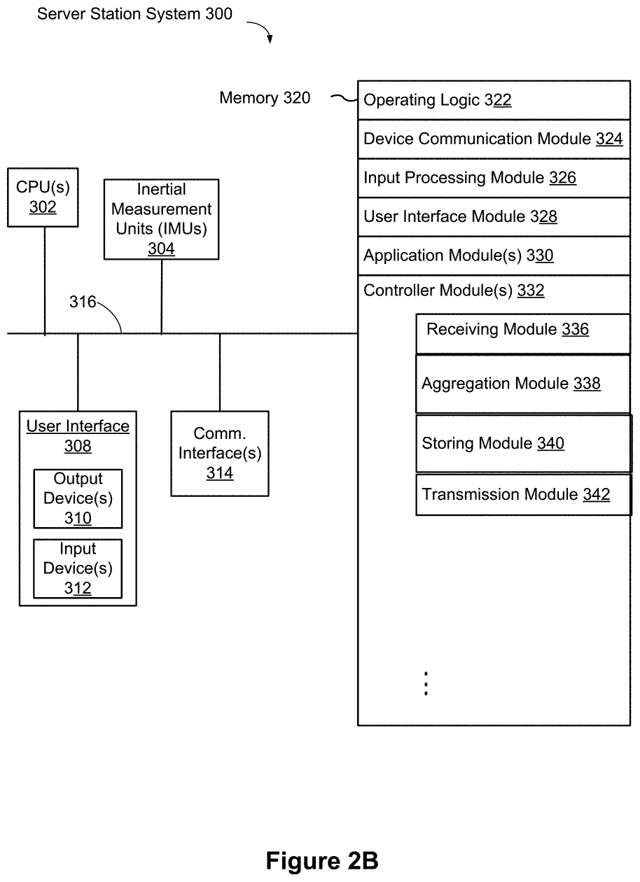

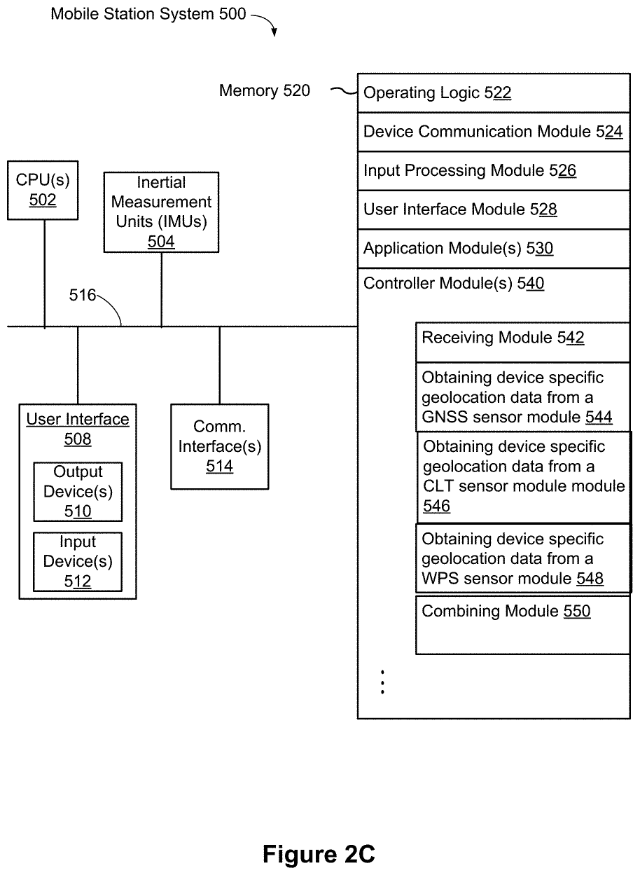

Referring to FIG. 2A there is shown a block diagram illustrating a representative base station system 200, according to some implementations.

In some implementations, the system 200 includes one or more processing units (e.g., CPUs, ASICs, FPGAs, microprocessors, and the like) 202, one or more communication interfaces 214, memory 220, and one or more communication buses 216 for interconnecting these components (sometimes called a chipset). The type of processing units 202 is chosen to match the requirement of application, including power requirements, according to some implementations. For example, the speed of the CPU should be sufficient to match application throughput.

In some implementations, the system 200 includes a user interface 208. In some implementations, the user interface 208 includes one or more output devices 210 that enable presentation of media content, including one or more speakers and/or one or more visual displays. In some implementations, user interface 208 also includes one or more input devices 212, including user interface components that facilitate user input such as a keyboard, a mouse, a voice-command input unit or microphone, a touch-screen display, a touch-sensitive input pad, a gesture-capturing device, or other input buttons or controls. Furthermore, some systems use a microphone and voice recognition or a camera with gesture recognition or a motion device with gesture recognition to supplement or replace the keyboard.

In some implementations, the system 200 includes one or more inertial measurement unit(s) (IMUs) 204. In some implementations, the IMUs include one or more accelerometers, one or more magnetometers, and/or one or more gyroscopes, and/or altimeters, and/or pressure sensors.

Communication interfaces 214 include, for example, hardware capable of data communications using any of a variety of custom or standard wireless protocols (e.g., IEEE 802.15.4, Wi-Fi, ZigBee, 6LoWPAN, Thread, Z-Wave, Bluetooth Smart, ISA100.11a, WirelessHART, MiWi) and/or any of a variety of custom or standard wired protocols (e.g., Ethernet, HomePlug), or any other suitable communication protocol, including communication protocols not yet developed as of the filing date of this document. In some implementations, communication interface 214 includes a global navigation satellite system, cellular network connectivity or wi-fi connectivity.

Memory 220 includes high-speed random access memory, such as DRAM, SRAM, DDR RAM, or other random access solid-state memory devices; and, optionally, includes non-volatile memory, such as one or more magnetic disk storage devices, one or more optical disk storage devices, one or more flash memory devices, one or more EPROMs, one or more EEPROMs, or one or more other non-volatile solid-state storage devices. Memory 220, or alternatively the non-volatile memory within memory 220, includes a non-transitory computer readable storage medium. In some implementations, memory 220, or the non-transitory computer readable storage medium of memory 220, stores the following programs, modules, and data structures, or a subset or superset thereof:
operating logic 222 including procedures for handling various basic system services and for performing hardware dependent tasks;
device communication module 224 for connecting to and communicating with other network devices (e.g., network interface, such as a router that provides Internet connectivity, networked storage devices, network routing devices, server system) connected to one or more networks via one or more communication interfaces 214 (wired or wireless);
input processing module 226 for detecting one or more user inputs or interactions from the one or more input devices 212 and interpreting the detected inputs or interactions;
user interface module 228 for providing and displaying a user interface in which settings, captured data, and/or other data for one or more devices (not shown) can be configured and/or viewed;
one or more application modules 230 for execution by the system 200 for controlling devices, and for reviewing data captured by devices (e.g., device status and settings, captured data, or other information regarding the system 200 and/or other client/electronic devices);
one or more controller modules 240, which provides functionalities for processing data from the one or more IMUs 204, including but not limited to:
   receiving module 242
   positioning module 244
   transmission module 246

In some implementations, the receiving module 242 is configured to receive geolocation data from satellites, Cellular Location Technology (CLT) geolocation data from cellular networks, Wi-Fi Positioning System (WPS/WiPS/WFPS) geolocation data from mobile hotspots or Wireless Access Points.

In some implementations, the positioning module 244 is configured to perform positioning error correction, and signal processing, on the cumulative geolocation data to form a high accuracy Global Positioning System (GPS). In some implementations, GPS includes the GNSS, CLT, WPS technologies readily available on or through cellphones.

In some implementations, the transmission module 246 is configured to transmit error corrections and processed GPS data to one or more servers coupled to the network.

Each of the above-identified elements may be stored in one or more of the previously mentioned memory devices and corresponds to a set of instructions for performing a function described above. The above-identified modules or programs (i.e., sets of instructions) need not be implemented as separate software programs, procedures, or modules, and thus various subsets of these modules may be combined or otherwise rearranged in various implementations. In some implementations, memory 220, optionally, stores a subset of the modules and data structures identified above. Furthermore, memory 220, optionally, stores additional modules and data structures not described above.

Referring to FIG. 2B there is shown a block diagram illustrating a representative server system, according to some implementations.

In some implementations, the system 300 includes one or more processing units (e.g., CPUs, ASICs, FPGAs, microprocessors, and the like) 302, one or more communication interfaces 314, memory 320, and one or more communication buses 316 for interconnecting these components (sometimes called a chipset). The type of processing units 302 is chosen to match the requirement of application, including power requirements, according to some implementations. For example, the speed of the CPU should be sufficient to match application throughput.

In some implementations, the system 300 includes a user interface 308. In some implementations, the user interface 308 includes one or more output devices 310 that enable presentation of media content, including one or more speakers and/or one or more visual displays. In some implementations, user interface 308 also includes one or more input devices 312, including user interface components that facilitate user input such as a keyboard, a mouse, a voice-command input unit or microphone, a touch-screen display, a touch-sensitive input pad, a gesture-capturing device, or other input buttons or controls. Furthermore, some systems use a microphone and voice recognition or a camera with gesture recognition or a motion device with gesture recognition to supplement or replace the keyboard.

In some implementations, the system 300 includes one or more IMUs 304. In some implementations, the IMUs include one or more accelerometers, one or more magnetometers, and/or one or more gyroscopes, and/or altimeters, and/or pressure sensors.

Communication interfaces 314 include, for example, hardware capable of data communications using any of a variety of custom or standard wireless protocols (e.g., IEEE 802.15.4, WiFi, ZigBee, 6LoWPAN, Thread, Z-Wave, Bluetooth Smart, ISA100.11a, WirelessHART, MiWi) and/or any of a variety of custom or standard wired protocols (e.g., Ethernet, HomePlug), or any other suitable communication protocol, including communication protocols not yet developed as of the filing date of this document. In some implementations, communication interface 314 includes a global navigation satellite system, cellular network connectivity or Wifi connectivity.

Memory 320 includes high-speed random access memory, such as DRAM, SRAM, DDR RAM, or other random access solid-state memory devices; and, optionally, includes non-volatile memory, such as one or more magnetic disk storage devices, one or more optical disk storage devices, one or more flash memory devices, one or more EPROMs, one or more EEPROMs, or one or more other non-volatile solid-state storage devices. Memory 320, or alternatively the non-volatile memory within memory 320, includes a non-transitory computer-readable storage medium. In some implementations, memory 320, or the non-transitory computer-readable storage medium of memory 320, stores the following programs, modules, and data structures, or a subset or superset thereof:
operating logic 322 including procedures for handling various basic system services and for performing hardware dependent tasks;
device communication module 324 for connecting to and communicating with other network devices (e.g., network interface, such as a router that provides Internet connectivity, networked storage devices, network routing devices, server system) connected to one or more networks via one or more communication interfaces 314 (wired or wireless);
input processing module 326 for detecting one or more user inputs or interactions from the one or more input devices 312 and interpreting the detected inputs or interactions;
user interface module 328 for providing and displaying a user interface in which settings, captured data, and/or other data for one or more devices (not shown) can be configured and/or viewed;
one or more application modules 330 for execution by the system 300 for controlling devices, and for reviewing data captured by devices (e.g., device status and settings, captured data, or other information regarding the system 300 and/or other client/electronic devices);
one or more controller modules 340, which provides functionalities for processing data from the one or more IMUs 304, including but not limited to:
   receiving module 336
   aggregation module 338
   storing module 340
   transmission module 342.

In some implementations, receiving module 336 receives the GNSS, CLT, and Wi-Fi error corrections and processed GPS data.

In some implementations, aggregation module 338 aggregates the GNSS, CLT, and Wi-Fi error corrections and processed GPS data.

In some implementations, storing module 340 stores the GNSS, CLT, and Wi-Fi error corrections and processed GPS data.

In some implementations, transmission module 342 transmits the location-specific GPS data to any mobile device in proximity.

Each of the above-identified elements may be stored in one or more of the previously mentioned memory devices and corresponds to a set of instructions for performing a function described above. The above-identified modules or programs (i.e., sets of instructions) need not be implemented as separate software programs, procedures, or modules, and thus various subsets of these modules may be combined or otherwise rearranged in various implementations. In some implementations, memory 320, optionally, stores a subset of the modules and data structures identified above. Furthermore, memory 320, optionally, stores additional modules and data structures not described above.

Referring to FIG. 2C there is shown a block diagram illustrating a representative mobile device system, according to some implementations.

In some implementations, the system 500 includes one or more processing units (e.g., CPUs, ASICs, FPGAs, microprocessors, and the like) 502, one or more communication interfaces 514, memory 520, and one or more communication buses 516 for interconnecting these components (sometimes called a chipset). The type of processing unit 502 is chosen to match the requirement of the application, including power requirements, according to some implementations. For example, the speed of the CPU should be sufficient to match application throughput.

In some implementations, the system 500 includes a user interface 508. In some implementations, the user interface 508 includes one or more output devices 510 that enable presentation of media content, including one or more speakers and/or one or more visual displays. In some implementations, user interface 508 also includes one or more input devices 512, including user interface components that facilitate user input such as a keyboard, a mouse, a voice-command input unit or microphone, a touch-screen display, a touch-sensitive input pad, a gesture-capturing device, or other input buttons or controls. Furthermore, some systems use a microphone and voice recognition or a camera with gesture recognition or a motion device with gesture recognition to supplement or replace the keyboard.

In some implementations, the system 500 includes one or more IMUs 504. In some implementations, the IMUs include one or more accelerometers, one or more magnetometers, and/or one or more gyroscopes, and/or altimeters, and/or pressure sensors.

Communication interfaces 514 include, for example, hardware capable of data communications using any of a variety of custom or standard wireless protocols (e.g., IEEE 802.15.4, WiFi, ZigBee, 6LoWPAN, Thread, Z-Wave, Bluetooth Smart, ISA100.11a, WirelessHART, MiWi) and/or any of a variety of custom or standard wired protocols (e.g., Ethernet, HomePlug), or any other suitable communication protocol, including communication protocols not yet developed as of the filing date of this document. In some implementations, communication interface 514 includes a global navigation satellite system, cellular network connectivity or Wi-Fi connectivity.

Memory 520 includes high-speed random-access memory, such as DRAM, SRAM, DDR RAM, or other random-access solid-state memory devices, and, optionally, includes non-volatile memory, such as one or more magnetic disk storage devices, one or more optical disk storage devices, one or more flash memory devices, one or more EPROMs, one or more EEPROMs, or one or more other non-volatile solid-state storage devices. Memory 520, or alternatively the non-volatile memory within memory 520, includes a non-transitory computer-readable storage medium. In some implementations, memory 520, or the non-transitory computer-readable storage medium of memory 520, stores the following programs, modules, and data structures, or a subset or superset thereof:
operating logic 522, including procedures for handling various basic system services and for performing hardware-dependent tasks;
device communication module 524 for connecting to and communicating with other network devices (e.g., network interface, such as a router that provides Internet connectivity, networked storage devices, network routing devices, server system) connected to one or more networks via one or more communication interfaces 514 (wired or wireless);
input processing module 526 for detecting one or more user inputs or interactions from the one or more input devices 512 and interpreting the detected inputs or interactions;
user interface module 528 for providing and displaying a user interface in which settings, captured data, and/or other data for one or more devices (not shown) can be configured and/or viewed;
one or more application modules 530 for execution by the system 500 for controlling devices, and for reviewing data captured by devices (e.g., device status and settings, captured data, or other information regarding the system 500 and/or other client/electronic devices); and one or more controller modules 540, which provide functionalities for processing data from the one or more IMUs 504, including but not limited to:
   receiving module 542;
   obtaining device-specific geolocation data from a global navigation satellite system (GNSS) sensor module 544;
   obtaining device-specific geolocation data from a Cellular Location Technology (CLT) sensor module 546;
   obtaining device-specific geolocation data from a Wi-Fi Positioning System (WPS) sensor module 548; and
   combining module 550.

In some implementations, receiving module 542 receives the location-specific Global Positioning System (GPS) data from a Referential Global Positioning System (RGPS) server in proximity to the mobile device.

In some implementations, obtaining device-specific geolocation data from a GNSS sensor module 544 obtains device-specific geolocation data from a GNSS sensor on the mobile device.

In some implementations, obtaining device-specific geolocation data from a CLT sensor module 546 obtains device-specific geolocation data from a CLT sensor on the mobile device.

In some implementations, obtaining device-specific geolocation data from a WPS sensor module 548 obtains device-specific geolocation data from a WPS sensor on the mobile device.

In some implementations, combining module 550 combines the location-specific GPS data with the device-specific GPS data to calculate a an error corrected location (e.g., <5 m) location of the mobile device.

Each of the above-identified elements may be stored in one or more of the previously mentioned memory devices and corresponds to a set of instructions for performing a function described above. The above-identified modules or programs (i.e., sets of instructions) need not be implemented as separate software programs, procedures, or modules, and thus various subsets of these modules may be combined or otherwise rearranged in various implementations. In some implementations, memory 220, optionally, stores a subset of the modules and data structures identified above. Furthermore, memory 220, optionally, stores additional modules and data structures not described above.

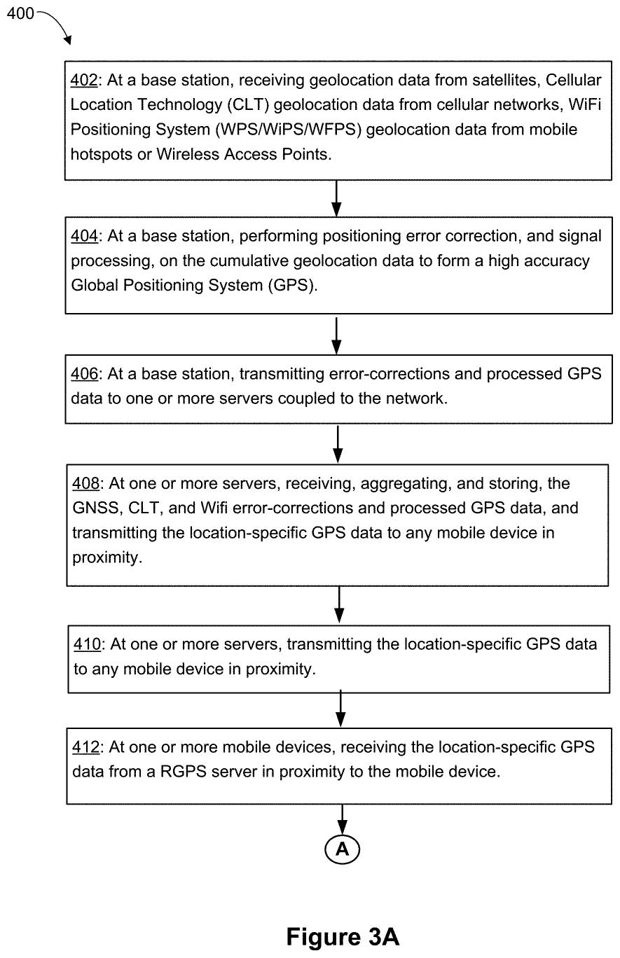

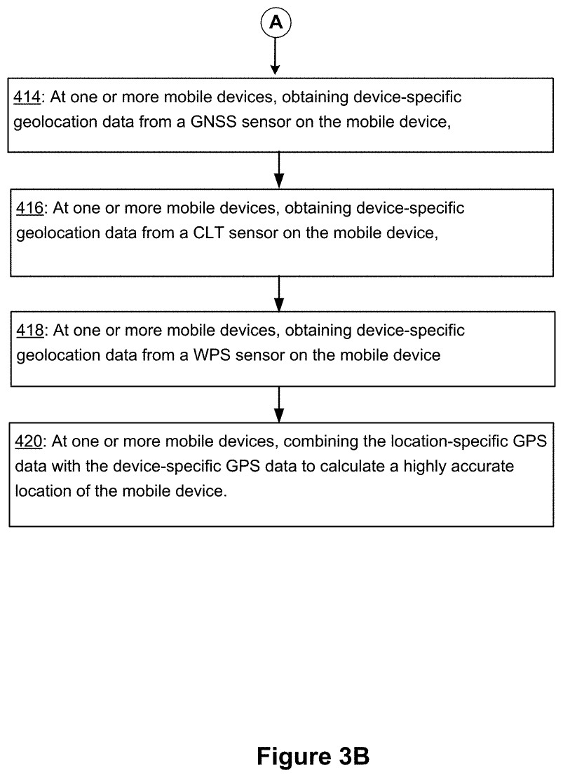

Referring to FIGS. 3A-3B, there is shown a flowchart representation of a method 400 performed by one or more of the components of system 100 designed to calculate an (accurate) error corrected location of a mobile device.

In some implementations, a plurality of RGPS base stations may be connected via a network.

At step 402, the method 400 may include, at the base stations that may include one or more GNSS receivers and antennas, receiving geolocation data from satellites, CLT geolocation data from cellular networks, and Wi-Fi Positioning System (WPS/WiPS/WFPS) geolocation data from mobile hotspots or Wireless Access Points.

At step 404, the method 400 may include, at the base stations that may include a data processor, performing positioning error correction, and signal processing, on the cumulative geolocation data to form a high-accuracy GPS.

At step 404, the method 400 may include, at the base stations that may include a transmitter, transmitting error corrections and processed GPS data to one or more servers coupled to the network.

In some implementations, each RGPS base station is configured to receive the GNSS signal data from a plurality of satellites, calculate coordinates for a location, and compare the calculated coordinates to predetermined coordinates to generate a compensation value or an error-correction data for the location.

In some implementations, each RGPS base station is configured to receive the CLT signal data from a plurality of cellular network signals, calculate coordinates for a location, and compare the calculated coordinates to predetermined coordinates to generate a compensation value or an error-correction data for the location.

In some implementations, each RGPS base station is configured to receive the WPS signal data from a plurality of network devices, calculate coordinates for a location, and compare the calculated coordinates to predetermined coordinates to generate a compensation value or an error-correction data for the location.

In some implementations, each RGPS base station is further configured to receive the GNSS, CLT, and WPS signal data, calculate the coordinates for the location, and compare the calculated coordinates to predetermined coordinates on a continuous and periodic basis.

In some implementations, each RGPS base station is further configured to receive the GNSS, CLT, and WPS signal data, calculate the coordinates for the location, and compare the calculated coordinates to predetermined coordinates, for different pre-determined times of a day.

At step 408, the method 400 may include, at one or more servers, receiving, aggregating, and storing, the GNSS, CLT, and Wi-Fi error corrections and processed GPS data.

At step 410, the method 400 may include, at one or more servers, transmitting the location-specific GPS data to any mobile device in proximity.

In some implementations, the one or more servers or the plurality of RGPS base stations include a wireless communication subsystem to wirelessly transmit the location-specific GPS data to any mobile device in proximity.

In some implementations, each RGPS base station is configured to transmit respective base station identification data that includes a respective identifier, a location, date and time of the error correction, or identifiers of location of the GNSS satellites, CLT network towers, and WPS network devices, and wherein the one or more servers are configured to receive and use the respective base station identification data to obtain the location-specific GPS data.

At step 412, the method 400 may include, at one or more mobile devices, receiving the location-specific GPS data from a RGPS server in proximity to the mobile device.

At step 414, the method 400 may include, at one or more mobile devices, obtaining device-specific geolocation data from a GNSS sensor on the mobile device.

At step 416, the method 400 may include, at one or more mobile devices, obtaining device-specific geolocation data from a CLT sensor on the mobile device.

At step 418, the method 400 may include, at one or more mobile devices, obtaining device-specific geolocation data from a WPS sensor on the mobile device.

At step 420, the method 400 may include, at one or more mobile devices, combining the location-specific GPS data with the device-specific GPS data to calculate an error corrected location of the mobile device.

In some implementations, generating an error corrected accurate location (e.g., highly accurate) may occur as described herein.
A) GNSS data may include:
1) determining actual geolocation through averaging over several hours or days (LGave),
2) reading GNSS satellite aggregate data output from sensor (LG),
3) reading GNSS satellite-specific geolocation data for each satellite (LGSn),
4) reading GNSS satellite-specific confidence value (LGCn),
5) calculating sum of GNSS confidence (LGCsum).
6) calculating average confidence (LGCave),
7) and calculating correction offset weights for each satellite signal (LWSn).
B) CLT Data may include (i) reading CLT data output from sensor=LC and (ii) reading CLT confidence value=LCc.
C) WPS Data may include (1) reading WPS data output from cellphone=LW
D) Correction weights on RGPS device or on server may include:
1) Determining confidence factor weight between L_{G} and L_{C} as follows:

WLGC=LGCave/(LGCave+LCc)

WLCG=1−WLGC
2) Optimizing weights for GNSS data (WLG), CLT data (WLC), and WPS data (WLW) using an optimization model (i.e., Levenberg-Marquardt) in the following balancing equation: LGave=WLG*(LG*WLGC)+WLC*(LC*WLCG)+WLW*(Lw)

Operations D1 and D2 described above may also be performed on a mobile device, wherein the device behaves as a mobile RGPS. The GNSS, CLT, and WPS data can be native or imported from a secondary RGPS node or server.
E) Data Correction on a mobile device may include:
1) reconstructing GNSS positioning from satellites available on a mobile device (LGM) with weighted offset correction:

LGM=ΣLGMn−{LWSn*[1−(|LGCMn−LGCMave|/LGCMsum)]}

LGM=LGM/number of satellites in view
2) Data Correction on a mobile device may include using the correction weights from (D) to calculate the high-accuracy GPS location (LM) on a mobile device:

LM=WLG*(LGM*WLMGC)+WLC*(LCM*WLMCG)+WLW*(LMW)

In some implementations, at least some of the RGPS base stations are positioned at locations where there is no reception or only partial reception of GPS signals from the respective networks, and wherein the locations are in proximity to predetermined locations of the one or more mobile devices.

In some implementations, the locations are at or near street-level close to streets where the one or more mobile devices are known to be present.

In some implementations, each mobile device includes a software application that (i) receives the location-specific GPS data, (ii) obtains the device-specific GPS location data, and (iii) combines the location-specific GPS data with the device-specific GPS data to calculate the location of the mobile device.

In some implementations, each mobile device includes a software application that (i) stores the location of the mobile device on the mobile device and (ii) provides the location to one or more other applications on the mobile device.

n some implementations, one or more mobile devices include a motion sensor with drift correction configured to track precision location and orientation of a mobile device regardless of external reference markers, transponders, or satellites, and the one or more mobile devices are further configured to fuse the precision location and orientation with the location-specific GPS data and the device-specific GPS data to calculate the location of the mobile device.

In some implementations, the one or more mobile devices are configured to use Bluetooth as a metric to determine proximity to another device in addition to GPS.

In some implementations, the one or more mobile devices are configured to use a geo-coding format for distancing queries and zone classification in three dimensions.

In some implementations, the one or more mobile devices are configured to use anonymous tokens or encoding that provides security, and the anonymous tokens include (i) a specification of resolution depth of geospatial data, (ii) an obfuscation or encryption for privacy or security that encrypts any address-specific component, and/or (iii) unencrypted data for number of interactions within a region, times of data, or demographic data.

In some implementations, the one or more servers or the one or more mobile devices are configurable to program or control variables or policies for minimum distancing, exposure time, or demographic data during a real-world event.

In some implementations, the one or more servers or the one or more mobile devices are configured to generate visualizations for contact tracing in a mobile application, showing historical interactions with risky individuals, risk levels, or courses of action.

In some implementations, the software application is further configured to establish a data connection with the one or more servers, transmit an approximate or non-corrected GPS location of the mobile device to the one or more servers, and receive a relevant error correction and processed GPS data generated by a base station that is most proximate to the mobile device's current location.

In some implementations, each mobile device is further configured to transmit the location of the mobile device to the one or more servers, and the one or more servers is further configured to receive the location of the mobile device to update the location-specific GPS data.

In some implementations, the one or more servers are further configured to (i) receive the location of the mobile device from the mobile device, and (i) track locations of the one or more mobile devices based on the location of the mobile device.

In some implementations, the one or more servers are further configured to generate an alarm if it is determined that any of the one or more mobile devices are closer than a predetermined distance.

In some implementations, the one or more servers are further configured to generate and transmit an alarm to the one or more mobile devices, or to a third-party device distinct from the one or more mobile devices, if it is determined that any of the one or more mobile devices are close to a predetermined location.

In some implementations, the one or more servers are further configured to anonymize any identifiable information received from the one or more mobile devices before transmitting any notifications to the one or more mobile devices, or a third-party device distinct from the one or more mobile devices.

In some implementations, the one or more servers are further configured to track locations of the one or more mobile devices to determine a set of devices of the one or more mobile device that may be within a given proximity to another device.

In some implementations, position re-enforcement for the mobile device may be performed using Bluetooth receiver strength signal indicator.

In some implementations, the location data calculated on mobile device will also include Bayesian mechanics.

In some implementations, calculations may include additional factors such as geodetic corrections based on observed satellite look angles.

### Geospatial Technology for Disease Prevention

In some implementations, such implementations generally relate to the field of producing and storing anonymous geolocation data with travel paths on mobile devices for use in conjunction with collected symptomatic census data, for disease prevention during pandemics.

Such implementations will be referred to hereafter as BIOS.

In some implementations, BIOS is a system capable of producing and storing anonymous geolocation data with travel paths on mobile devices, conjunctively with collected symptomatic census data, for disease prevention during pandemics. The principal objective of BIOS is to increase disease prevention velocity to a threshold above infection velocity by means of technology to subvert a given infectious disease out of populous at an accelerated rate. Using a fusion of GPS, inertial sensors, personal assessment, public health databases, and health expert input creates an integrated biokinetic tool for society, health officials, and governments to help optimize public policies and resource allocation in order to stay ahead of disease infection rate while minimizing economic and psychological impact on public. Individuals suspected of infection may permit officials to produce an instantaneous historical interaction graph with geolocation to identify other individuals at high, medium, and low risk of infection. Notifications can be sent instantaneously to individuals at risk, with effective medical response in high-risk zones. Predefined minimum distances can be programmed in BIOS to provide annunciations or alarms on mobile devices if any individuals approach one another within the prescribed minimum distances.

In the absence of immunological solutions such as vaccines or other therapeutic measures, only two competing factors control the spread of an outbreak: velocity of infection versus the velocity of prevention. Decisions on forming public policies for government officials are complex and made more difficult by hidden or unknown characteristics of the pathogen in the early stages of a pandemic or epidemic. Velocity of prevention becomes dependent on public health advisory, restrictions, and behavioral response of society within the regional scope of public policies. Initial lack of data on behavior of a disease limits the accuracy of base assumptions applied to prediction models. Refinement of the base assumptions is accomplished by implementing iterations of preventative public policies and observing the effects of such policies on the spread of the disease. A drawback in these circumstances is that the disease inevitably spreads, until the base assumptions are updated after weeks and months of collected data to more effectively subdue the velocity of infection.

In some implementations, most often proximity and duration of interactions between individuals in society, a function of normal human behavior, catalyzes infection rates. Studying these behavioral patterns can assist in designing response policies that can effectively stop the spread of a pathogen. Cellular phones are commonly used worldwide and act as an immediate source for gathering data on human biokinetic behavior. Geolocation and distance measurements can be performed on mobile devices rapidly and stored for analysis in conjunction with an individual's health condition, providing datasets for medical analysis and base-assumption corrections at a high rate. Demographic-related data can be combined with places globally, near real-time, leading to faster and more accurate decisions on preventative public policies by government officials. Recent research has suggested that the use of technology can greatly enhance disease prevention during pandemics (L. Ferretti et al.).

In some implementations, current attempts at contract-tracing by big-data companies involve use of limited and inaccurate methods of distance measurement. A complex set of communication exchange protocols, designed to operate over Bluetooth Low Energy (BLE), is used as the basis of measurement and communication between users. Such methods are anticipated to be ineffective and have already proved to be insecure (Celosia et al.). Excessive indulgence at user-side anonymity, while lacking basic security, minimizes the disease prevention potency of technology-based countermeasures. No intelligible information on the infectious disease can be had by authorities to help lower the velocity of infection.

In some implementations, BIOS implements a simplified token with sufficient user-side anonymity as well as a server-side secure token, capable of generating regional-level intelligence on characteristics of an infectious disease. Live reporting enabled by BIOS gives medical and government officials tools to more effectively analyze, recommend, and build better policies very quickly. A proper implementation of readily available technology can greatly decrease the velocity of infection and, in so doing, increase the velocity of prevention.

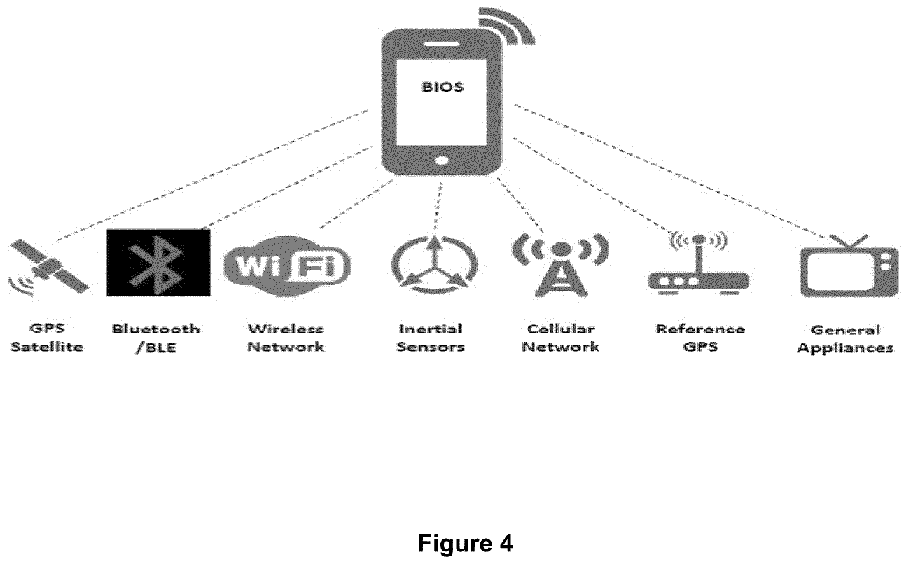

FIG. 4 illustrates a BIOS system according to some implementations. In some implementations, BIOS leverages multiple methods of generating geolocation data that are commonly available on mobile devices, such as cellular phones. Additionally, BIOS introduces the concept of Reference GPS (RGPS) to improve accuracy of standard cellular GPS on mobile devices. RGPS, a patent-pending technology, works similarly to Differential GPS (DGPS), with a significant difference in base station data and its utilization by the mobile device. By consuming multiple types of geolocation signals, BIOS can locate a user with much greater accuracy when compared with conventional systems, enabling particularly precise measurements of social distancing and biokinetic interactions. Available measurement methods range from raw GPS, to cellular network signal (3G, 4G, LTE, 5G, etc.), to other radiometric techniques, such as Bluetooth and Wi-Fi. Common appliance signals can also be useful for position improvement calculations. If only a subset of these sources is operational, BIOS optimizes position estimation based on the available signal(s). The position estimates are stored on a user's mobile device and uploaded to regulated servers with end-to-end encryption in a unique geocoding format.

In some implementations, BIOS can use multiple sources of location information. More than one source enables better estimations on device location. New technology, RGPS, with base stations can be deployed throughout a city to provide multiple redundant corrections for all mobile device users, improving location accuracy.

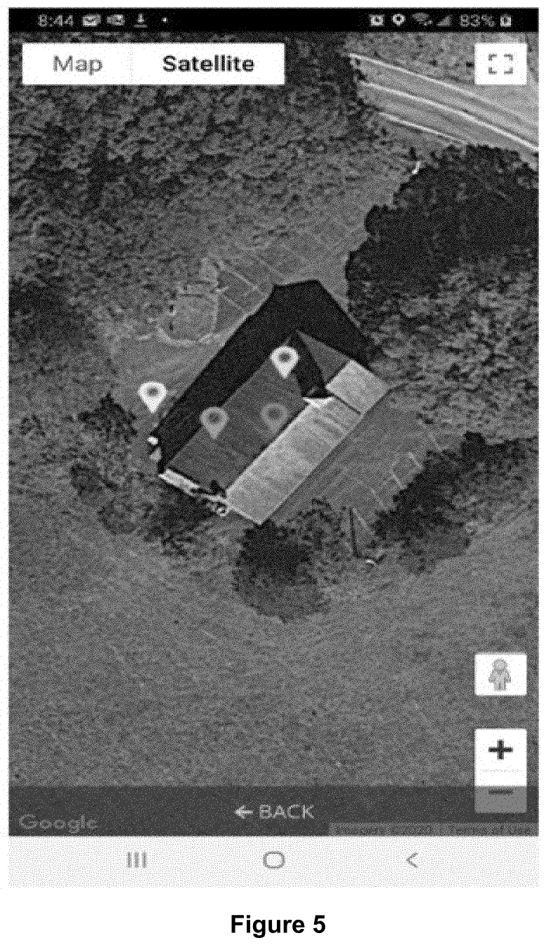

Preliminary testing of BIOS location measurements using multiple data sources was performed within the app. FIG. 5 illustrates various measurement sources, individually marked in different colors, on a Google Maps screen capture of the BIOS app. The green marker represents the raw GPS location as determined by the GPS sensor in the cellphone. The blue marker represents the location of the mobile device as determined by the cellular network signal (4G). The yellow marker represents the location of a nearby base station. RGPS data is provided to the BIOS app internally and used, along with other aforementioned sources, to estimate the corrected position of the mobile device indoors to a meter accuracy, which is represented by the red marker (behind the green marker).

In some implementations, a geocoding format that greatly simplifies distancing queries and zone classification in three dimensions is crucial. Current geocoding mechanisms, such as conventional GEOHASH, provide a good solution for two-dimensional distancing and zone classification; however, it lacks the elevation (or altitude) data in geospatial zone classification. In some implementations, GEOHASH may be modified to include altitude, in addition to longitude and latitude, and may be referred to herein as “GEOSHASH” (GEO‘S’HASH with an ‘S’ in the middle as opposed to GEOHASH). GEOSHASH provides a more efficient alternative for encoding three-dimensional zones. BIOS app uses GEOSHASH to generate a three-dimensional geolocation encoding during interactions.

In some implementations, GEOSHASH is an ordered alpha-numeric string compiled using a simplified GEOS base-64 method, encoding quadrature zone classification of the Earth (see Table 1). The encoded string can be of any length; the greater the length, the more precisely a volumetric zone is encoded. Objects occupying the same geospatial region will share identical GEOSHASH encoding, making database queries for objects in any given zone simple and fast. As an example, given two objects A and B, provided A is deemed to be positioned at a GEOSHASH of “SIMqW3b2Q9Tv,” and provided B is deemed to be positioned at a GEOSHASH of “SIMqW3b2Q9Tn,” both objects occupy the same geospatial zone of “SIMqW3b2Q9T” and are separated by approximately one meter with the resolution of GEOSHASH string containing 12 characters {String “SIMqW3b2Q9Tv”.length( )}.

TABLE 1
Comparison of the 2D GEOHASH vs. 3D GEOSHASH (from Geospatial Operating System)

| HASH | DIM | LEN | ENCODED TEXT | LATITUDE | LONGITUDE | ALTITUDE |
|---|---|---|---|---|---|---|
| GEOHASH-32 | 2D | 9 | dpz838bhd | 43.6426° | −79.3871° | NONE |
| GEOSHASH-32 | 3D | 15 | f4kv901t89n9fhx | 43.6426° | −79.3871° | 0.633 km |
| GEOSHASH-64 | 3D | 11 | SIMqW3b2Q9T | 43.6426° | −79.3871° | 0.633 km |

In some implementations, using the GEOSHASH-64 format, altitude can be encoded with two extra characters, compared to Base32 GEOHASH encoding, to support a 2,000 km range in the third, vertical dimension.

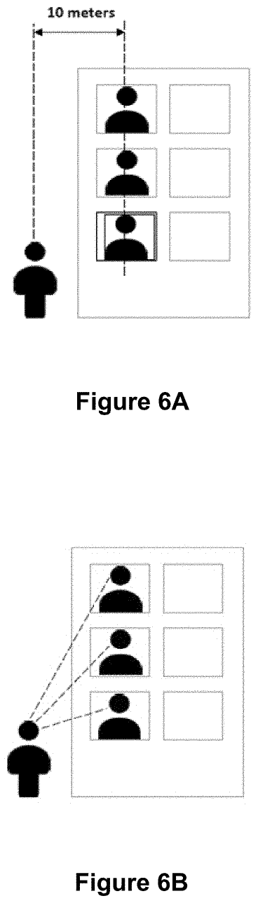

FIG. 6A illustrates a representation of how GEOHASH encodes only two dimensions according to some implementations. All three building occupants register the same longitude and latitude on ground. Extra calculation is required for altitude to determine the third-dimension distance. The distance from a person standing outside the building to all individuals inside the building registers as the same distance on the 2D GEOHASH.

FIG. 6B illustrates a representation of how GEOSHASH encodes location in three dimensions preventing false 2D-distance reports, providing true volumetric geospatial zoning according to some implementations. Each building occupant registers a different GEOSHASH. Distance from a person standing outside the building to all individuals inside the building register as distinct values on the 3D GEOSHASH.

In some implementations, GEOSHASH encoding provides an added dimension of security for users, while simultaneously provisioning geolocation visibility for government-use cases during outbreaks. Government officials may choose to specify resolution depth of geospatial data available for census and epidemic tracking purposes while the remainder of the GEOSHASH can be obfuscated or encrypted for privacy and security of the individual. As an example, authorities may choose to track a given disease and its propagation characteristics within a city, county, or state while protecting the address-specific locale of each user. In such a use-case, leaving the first seven characters (left to right) of GEOSHASH unencrypted will suffice for the census collection objectives of the government, while the remaining five characters may be encrypted by the user to maintain privacy, attempting to strike a balance between overall societal objective of health verses the individual's rights as it relates to geospatial data. An individual may voluntarily disclose address-specific geospatial data by unlocking the GEOSHASH encryption, using his or her BIOS passcode, if the need arises.

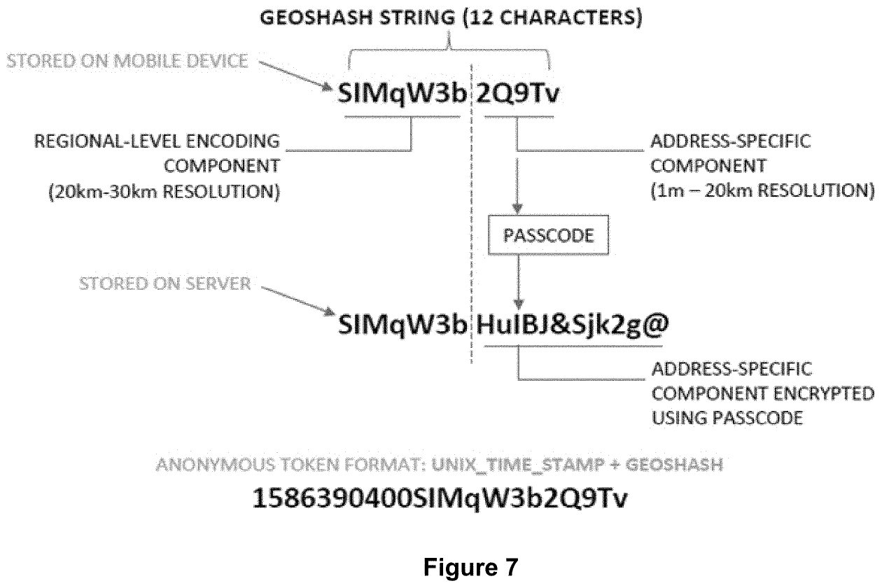

FIG. 7 illustrates a representation of a sample GEOSHASH string with its resolution components according to some implementations. Government may access GEOSHASH tags on a regional level to gather census data during pandemics or epidemics, while maintaining individual geospatial privacy. Information such as number of high-risk or low-risk interactions within a region, times of day, as well as demographic data can be accessed without compromising data privacy of the individual(s). Address-specific component of the GEOSHASH may be encrypted with a user's secret passcode for server storage. Users may decrypt the address-specific component voluntarily, if necessary. The anonymous token consists of an open or encoded form of Unix Timestamp concatenated with the GEOSHASH string.

In some implementations, BIOS provides an interface by which government officials or authorized medical personnel may program and deploy variables for minimum distancing, exposure time per distance, and relevant demographic data during a given pandemic or epidemic to mobile devices running the app. BIOS will begin collecting anonymous geotagged behavioral data based on such variables or assumptions. Continuous analysis can be performed to track the results of preliminary assumptions in real time. Corrections can be made to the tracking system's variables on a day-to-day basis rather than weeks or months, enabling better intelligence over the disease and leading to increased velocity of prevention while decreasing infection rates.

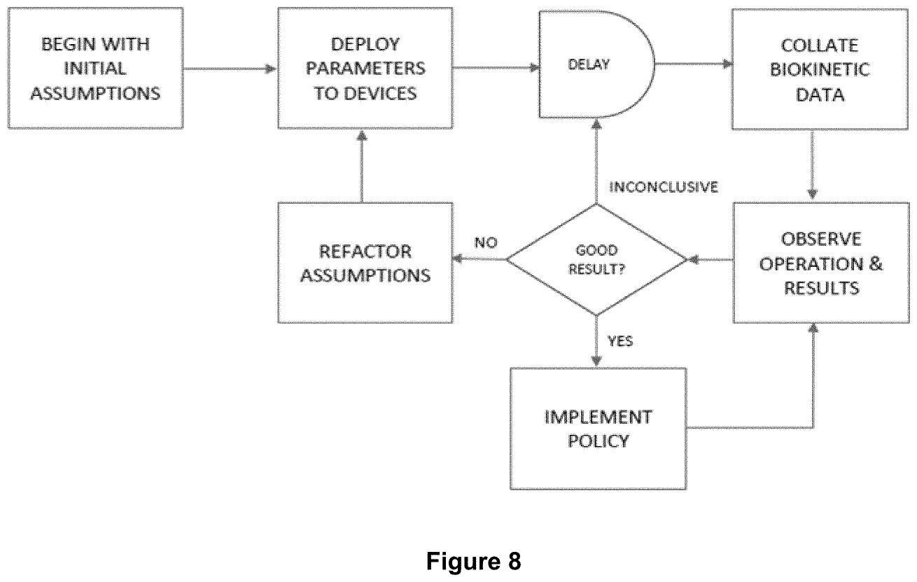

FIG. 8 illustrates a representation of a BIOS pandemic prevention mechanism according to some implementations. The BIOS pandemic prevention mechanism begins with gathering initial assumptions about the characteristic behavior of a disease and deploying the parameters to mobile devices. Performance of the initial assumptions is observed, and the assumptions are refactored for the next cycle of deployment back to the mobile device via the BIOS app in a continuous loop, converging to the most optimal values that effectively control the disease. Public policies can now be established for a period to determine the declination rate of the disease. If the policy holds true, then the policy continues until disease is under control, otherwise assumptions may be refactored further.

In some implementations, BIOS is flexible, providing a system for authorities to carefully design and implement a robust disease prevention strategy. Three levels of default social distancing can be programmed by authorized medical personnel and government officials, while more custom parameters can be added to the system. The distance classifications are as follows:
(1) Dₗᵣ—Minimum Low-Risk Distance
(2) Dₘᵣ—Minimum Medium-Risk Distance
(3) Dₕᵣ—Minimum High-Risk Distance

Furthermore, temporal exposure variables are also provided for each distance risk level:
(1) Tₗᵣ—Maximum Low-Risk Distance Time
(2) Tₘᵣ—Maximum Medium-Risk Distance Time
(3) Tₕᵣ—Maximum High-Risk Distance Time

In some implementations, a cumulative sum of exposure time and distance is used to evaluate the risk factor for a user. Notifications are generated on the user's mobile device, alerting the individual while approaching increasing risk zones. Local logs are generated for the duration of the interaction, and the server is updated with encrypted GEOSHASH tags from each person's mobile device.

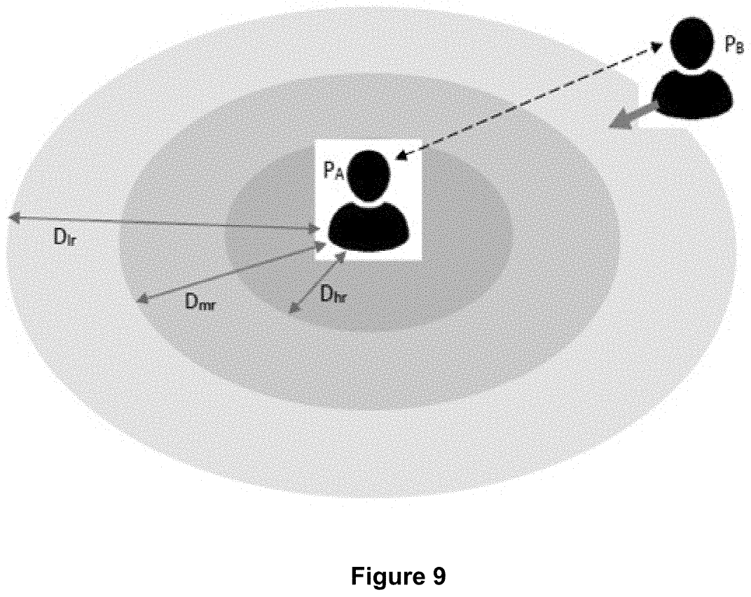

FIG. 9 illustrates a representation of an interaction between users according to some implementations. The interaction begins with a user (PB) entering minimum low-risk distance (Dir) to another individual (PA). Distance evaluation is provided by BIOS, triggering an alert on the mobile device of PA and PB of approaching each other. Onset of an interaction triggers data logging of the events every 10-20 seconds. Encrypted GEOSHASH tags are uploaded to the server and local data logging continues until users move away from one another, past the minimum low-risk distance.

In some implementations, on the mobile device a user can view the activity logs with High-Risk (red), Medium-Risk (amber), and Low-Risk (green) interactions over the past 60 days.

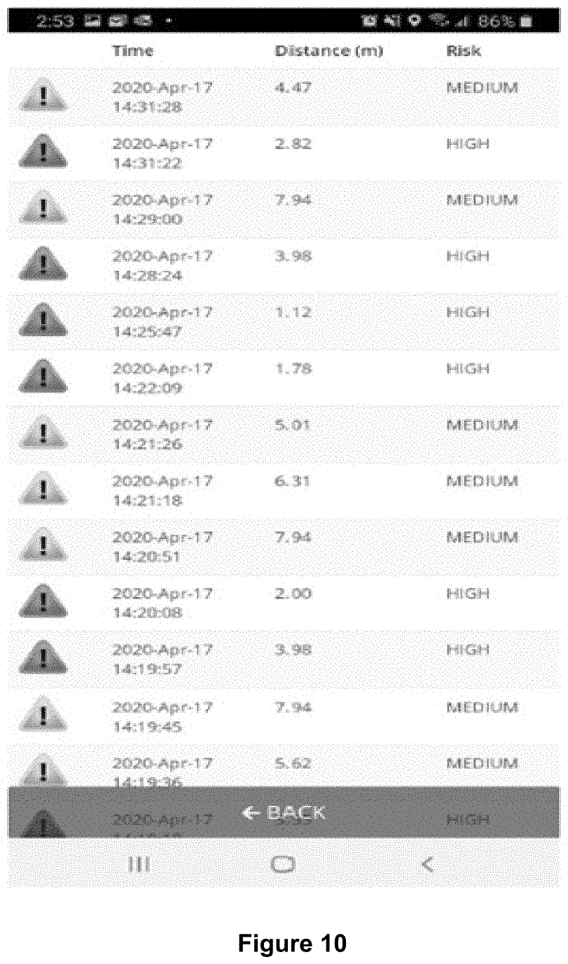

FIG. 10 is an image of a screen capture of the BIOS mobile app's Interactions Screen according to some implementations. The BIOS mobile app's Interactions Screen may display the historical interactions with other individuals in High-Risk (red) and Medium-Risk (amber) category. Risk levels determine the course of action to be taken, depending on government policies, if an individual with prior interactions is determined to be infected.

In some implementations, BIOS employs two storage mechanisms: local storage on the mobile device and encrypted database storage on edge servers. Locally stored data is copied to a server when an internet connection or cellular data access is available. Transfer of data is handled with end-to-end encryption over SSL. Upon receiving data, the server encrypts and stores user information in a protected database with field-level encryption on sensitive information such as email addresses, phone numbers, or any other communication channel provided by the user. The communication channels will be used for notification if the user has been determined to be at probable exposure to a disease.

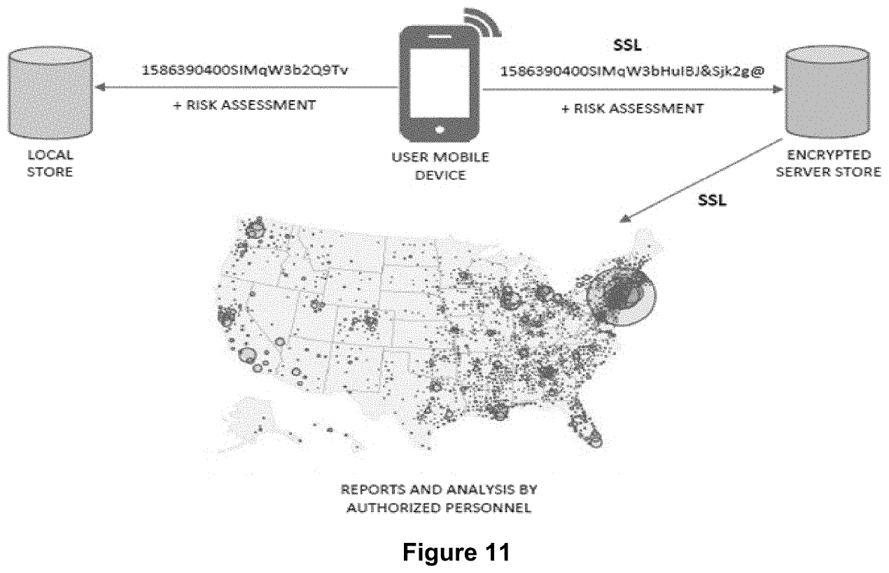

FIG. 11 illustrates a representation of how interaction data is utilized by a mobile device according to some implementations. For example, interaction data collected by a user's mobile device is used to create a local shared token that also identifies others within the same interaction event. A secure token with address-specific data encryption is sent to the server with risk assessment. Server-side data is also used for analysis by qualified personnel to improve prediction models and implement policies.

If an infectious event occurs, medical personnel can trigger notifications to other individuals who have been in contact or close proximity with the infected patient. The anonymous token array of interactions is pushed to the server, which in turn notifies all mobile devices to check if a matching anonymous token resides in their local mobile database. If a match is found, the user is immediately notified of the interaction risk level associated and stored in memory of the device. Subsequent queries made to the server by authorized medical personnel or government officials will reflect the updated regional statistics of the disease.

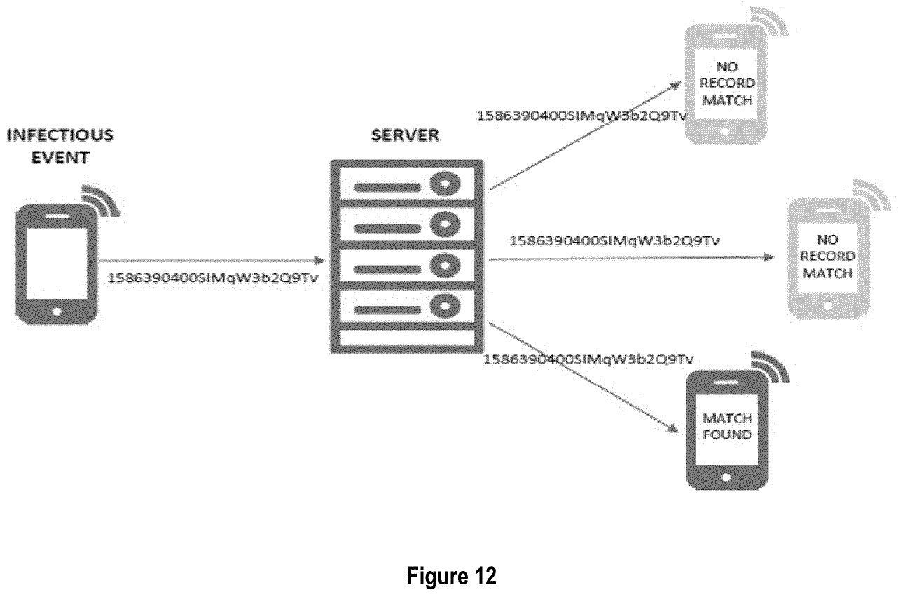

FIG. 12 illustrates a representation of how event data is transmitted between components of the BIOS system according to some implementations. For example, when an infectious event occurs, the local interaction tokens are transmitted to the server, where the token is broadcasted to mobile devices. When a matching token is found, the user of the matching mobile device is notified of the risk level, already stored on the user's device. Additional communication channels may be used to inform the individual with the matching interaction token of the potential exposure, and health assessment. The interaction token carries a locale signature that can be used to identify facilities or places that may have been contaminated for disinfection.

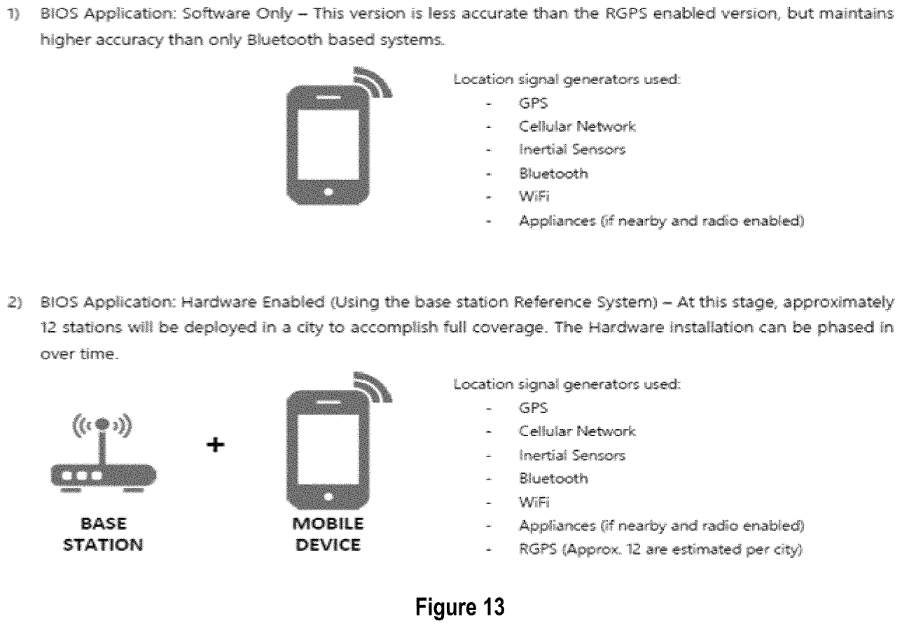

In some implementations, the BIOS app can work with various signals to identify user location in order to determine distancing and geolocation classification with GEOSHASH. Only the base station (RGPS signal requires additional hardware. Deployment can take place in two phases (as shown in FIG. 13):
(1) BIOS Application: Software Only—This version is less accurate than the RGPS-enabled version, but maintains higher accuracy than only Bluetooth-based systems.
(2) BIOS Application: Hardware Enabled (Using the base station RGPS)— At this stage, approximately 12 stations will be deployed in a city to accomplish full coverage. The Hardware installation can be phased in over time.

In some implementations, anonymous geolocation-based tracing of human interactions within a society, through the use of readily available technologies, can greatly benefit communities during episodes of aggressive pandemics and epidemics. Using multiple sources of geolocation data is essential for implementing a robust and highly accurate system. There are currently 3.5 billion smartphone users globally, making it the most commonly used personal device. Cellular phones incorporate sufficient technological frameworks to implement an effective disease prevention application in phase 1 without RGPS. Phase 2, with RGPS stations, can be introduced gradually for better general-purpose GPS positioning using smartphones. BIOS achieves a method of implementing geolocation technology for maximum velocity of disease prevention, while maintaining a user's right to privacy at a practical level for an optimum outcome in public health, that, in turn itself, is also a right of all individuals as participating members of society.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention.

It should be understood that the particular order in which the operations for methods described herein have been described is merely an example and is not intended to indicate that the described order is the only order in which the operations could be performed. One of ordinary skill in the art would recognize various ways to reorder the operations described herein.

Although some of various drawings illustrate a number of logical stages in a particular order, stages that are not order-dependent may be reordered and other stages may be combined or broken out. While some reordering or other groupings are specifically mentioned, others will be obvious to those of ordinary skill in the art, so the ordering and groupings presented herein are not an exhaustive list of alternatives. Moreover, it should be recognized that the stages could be implemented in hardware, firmware, software or any combination thereof.

The foregoing description, for purpose of explanation, has been described with reference to specific implementations. However, the illustrative discussions above are not intended to be exhaustive or limit the scope of the claims to the precise forms disclosed. Many modifications and variations are possible in view of the above teachings. The implementations were chosen in order to best explain the principles underlying the claims and their practical applications, to thereby enable others skilled in the art to best use the implementations with various modifications as are suited to the particular uses contemplated.

What is claimed is:

1. A system comprising:
a plurality of Referential Global Positioning System (RGPS) base stations that are connected via a network, wherein each RGPS station comprises (i) one or more global navigation satellite system (GNSS) receivers and antennas configured to receive GNSS geolocation data from satellites, Cellular Location Technology (CLT) geolocation data from cellular networks, and Wi-Fi Positioning System (WPS/WiPS/WFPS) geolocation data from mobile hotspots or Wireless Access Points to generate cumulative geolocation data; (ii) a data processor configured to perform positioning error correction, and signal processing, on the cumulative geolocation data to form a high-accuracy Global Positioning System (GPS); and (iii) a transmitter configured to transmit error corrections and processed GPS data to one or more servers coupled to the network;
the one or more servers configured to (i) receive, aggregate, and store the GNSS, CLT, and Wi-Fi error corrections and processed GPS data to form location-specific GPS data, and (ii) transmit the location-specific GPS data to any mobile device in proximity; and
one or more mobile devices, each mobile device configured to (i) receive the location-specific GPS data from an RGPS server in proximity to the mobile device, (ii) obtain device-specific geolocation data from a GNSS sensor on the mobile device, (iii) obtain device-specific geolocation data from a CLT sensor on the mobile device, (iv) obtain device-specific geolocation data from a WPS sensor on the mobile device and (v) combine the location-specific GPS data with the device-specific geolocation data to calculate a highly accurate location of the mobile device.

2. The system of claim 1, wherein at least some of the RGPS base stations are positioned at locations where there is no reception or only partial reception of GPS signals from the respective networks, and wherein the locations are in proximity to predetermined locations of the one or more mobile devices.

3. The system of claim 2, wherein the locations are at or near street-level close to streets where the one or more mobile devices are known to be present.

4. The system of claim 1, wherein the one or more servers or the plurality of RGPS base stations include a wireless communication subsystem to wirelessly transmit the location-specific GPS data to any mobile device in proximity.

5. The system of claim 1, wherein each RGPS base station is configured to receive the GNSS geolocation data from a plurality of satellites, calculate coordinates for a location, and compare the calculated coordinates to predetermined coordinates to generate a compensation value or an error-correction data for the location.

6. The system of claim 1, wherein each RGPS base station is configured to receive the CLT geolocation data from a plurality of cellular network signals, calculate coordinates for a location, and compare the calculated coordinates to predetermined coordinates to generate a compensation value or an error-correction data for the location.

7. The system of claim 1, wherein each RGPS base station is configured to receive the WPS geolocation data from a plurality of network devices, calculate coordinates for a location, and compare the calculated coordinates to predetermined coordinates to generate a compensation value or an error-correction data for the location.

8. The system of claim 1, wherein each RGPS base station is further configured to receive the GNSS, CLT, and WPS signal data, calculate the coordinates for the location, and compare the calculated coordinates to predetermined coordinates on a continuous and periodic basis.

9. The system of claim 1, wherein each RGPS base station is further configured to receive the GNSS, CLT, and WPS signal data, calculate the coordinates for the location, and compare the calculated coordinates to predetermined coordinates for different predetermined times of a day.

10. The system of claim 1, wherein each RGPS base station is configured to transmit respective base station identification data that includes a respective identifier, a location, date and time of the error correction, or identifiers of location of the GNSS satellites, CLT network towers, and WPS network devices, and wherein the one or more servers are configured to receive and use the respective base station identification data to obtain the location-specific GPS data.

11. The system of claim 1, wherein each mobile device includes a software application that (i) receives the location-specific GPS data, (ii) obtains the device-specific GPS location data, and (iii) combines the location-specific GPS data with the device-specific GPS data to calculate the location of the mobile device.

12. The system of claim 1, wherein the software application is further configured to establish a data connection with the one or more servers, transmit an approximate or non-corrected GPS location of the mobile device to the one or more servers, and receive a relevant error correction and processed GPS data generated by a base station that is most proximate to a current location of the mobile device.

13. The system of claim 1, wherein each mobile device includes a software application that (i) stores the location of the mobile device on the mobile device and (ii) provides the location to one or more other applications on the mobile device.

14. The system of claim 1, wherein each mobile device is further configured to transmit the location of the mobile device to the one or more servers, and wherein the one or more servers is further configured to receive the location of the mobile device to update the location-specific GPS data.

15. The system of claim 1, wherein the one or more servers are further configured to (i) receive location of the mobile device from the mobile device, and (i) anonymously track locations of the one or more mobile devices based on the location of the mobile device.

16. The system of claim 1, wherein the one or more servers are further configured to generate an alarm if it is determined that any of the one or more mobile devices are closer than a predetermined distance.

17. The system of claim 1, wherein the one or more servers are further configured to generate and transmit an alarm to the one or more mobile devices or to a third-party device distinct from the one or more mobile devices, if it is determined that any of the one or more mobile devices are close to a predetermined location.

18. The system of claim 1, wherein the one or more servers are further configured to anonymize any identifiable information received from the one or more mobile devices before transmitting any notifications to the one or more mobile devices or a third-party device distinct from the one or more mobile devices.

19. The system of claim 1, wherein the one or more servers are further configured to track locations of the one or more mobile devices to determine a set of devices of the one or more mobile device that may be within a given proximity to another device.

20. A method comprising:
at a plurality of RGPS base stations (e.g., some static, dynamic mobile station) that are connected via a network:
receiving, via one or more GNSS receivers and antennas, geolocation data from satellites, CLT geolocation data from cellular networks, and Wi-Fi Positioning System (WPS/WiPS/WFPS) geolocation data from mobile hotspots or Wireless Access Points to generate cumulative geolocation data,
performing, via a data processor, positioning error correction and signal processing on the cumulative geolocation data to form a high-accuracy GPS,
transmitting, via a transmitter, error corrections and processed GPS data to one or more servers coupled to the network;
at one or more servers:
receiving, aggregating, and storing, the GNSS, CLT, and Wi-Fi error-corrections and processed GPS data to form location-specific GPS data, and
transmitting the location-specific GPS data to any mobile device in proximity; and
at one or more mobile devices:
receiving the location-specific GPS data from an RGPS server in proximity to the mobile device,
obtaining device-specific geolocation data from a GNSS sensor on the mobile device,
obtaining device-specific geolocation data from a CLT sensor on the mobile device,
obtaining device-specific geolocation data from a WPS sensor on the mobile device, and
combining the location-specific GPS data with the device-specific GPS data to calculate a highly accurate location of the mobile device.
